(19) United States
(12) Patent Application Publication
(10) Pub. No.: US 2008/0003245 A1
(43) Pub. Date: Jan. 3, 2008

(54) Use of octyl salicylate in cosmetic preparations containing 1,3-dihydroxyacetone

(75) Inventors: Rainer Kroepke, Schenefeld (DE); Stephanie Von Der Fecht, Schenefeld (DE); Alexander Filbry, Hamburg (DE); Astrid Heptner, Hamburg (DE); Jens Schulz, Schenefeld (DE)

Correspondence Address: GREENBLUM & BERNSTEIN, P.L.C., 1950 ROLAND CLARKE PLACE, RESTON, VA, 20191, US

(73) Assignee: BEIERSDORF AG, Hamburg (DE)

(21) Appl. No.: 11/477,414

(22) Filed: Jun. 30, 2006

(51) Int. Cl. A61K 8/37

(52) U.S. Cl. 424/401; 512/21

(57) ABSTRACT

The present invention relates to a combination of octyl salicylate and 1,3-dihydroxyacetone in cosmetic preparations.

The present invention relates to a combination of octyl salicylate and 1,3 dihydroxyacetone in cosmetic preparations.

The trend away from genteel pallor towards “healthy, sporty tanned skin” has been unbroken for years. In order to attain this, people expose their skin to solar radiation since this brings about pigment formation in the sense of melanine formation. However, the ultraviolet radiation of sunlight has a harmful effect on the skin. Besides the acute damage (sunburn), long-term damage, such as suffering from an increased risk of skin cancer in cases of excessive irradiation with light from the UVB region (wavelength: 280-320 nm), arises. Moreover, the excessive effect of UVB and UVA radiation (wavelength: 320-400 nm) leads to a weakening of the elastic and collagenic fibers of the connective tissue. This leads to numerous phototoxic and photoallergic reactions and results in premature skin ageing.

However, human skin can also be tanned artificially. The artificial tanning of the skin can be produced, e.g., externally with the aid of cosmetics and orally by taking carotenoids.

However, the artificial tanning of the skin that can be obtained by applying so-called self-tanners is much more popular. These compounds have as chemical structural characteristic keto or aldehyde groups in the vicinity of alcohol functions. These ketols or aldols chiefly belong to the sugar substance group. The most frequently used self-tanner is 1,3-dihydroxyacetone.

Self-tanning compounds can be reacted with the proteins and amino acids of the horny layer of the epidermis in the sense of a Maillard reaction, whereby polymers are produced via a reaction pathway not yet fully elucidated, which polymers give the skin a brownish color. This reaction is completed after 4 to 6 hours, the tan thus achieved cannot be washed off and is removed only with the normal exfoliation of the skin.

However, one drawback of the prior art is that the 1,3-dihydroxyacetone in cosmetic preparations disintegrates over time. The instability of 1,3-dihydroxyacetone in cosmetic preparations and the decomposition process that is mostly to be attributed to oxidation occur in particular in the case of an aqueous cosmetic. The decomposition products of the 1,3-dihydroxyacetone (e.g., hydroxymethylglyoxal, formaldehyde, glycolic acid, formic acid, acetic acid, pyruvic acid), in addition to coloring the preparation brown, also result in a reduction of the pH value of the same and in an unpleasant odor of the cosmetic formulation. The odor of the decomposition products of the 1,3-dihydroxyacetone generally perceived as pungent and intensive usually means that the consumer no longer uses the cosmetic (C. Aretz, R. Buczys, K. Buchholz, H. Driller, Euro Cosmetics, p. 32-36).

In the past there has been no lack of attempts to delay the decomposition of the 1,3-dihydroxyacetone in the preparations. Thus WO 95/22960 describes the use of cyclodextrins or DE 10135024 the use of certain sunscreens.

However, the object is usually simply to cover (mask) the decomposition odor with perfumes.

It was therefore surprising and not foreseeable for one skilled in the art that the defects of the prior art were eliminated by
a) The use of octyl salicylate for perfuming and/or improving odor of cosmetic preparations containing 1,3-dihydroxyacetone.
b) The use of octyl salicylate for masking odor and/or stabilizing odor of cosmetic preparations containing 1,3-dihydroxyacetone.
c) The use of octyl salicylate for perfuming, improving odor, masking odor and/or stabilizing odor of cosmetic preparations containing 1,3-dihydroxyacetone and characterized in that the cosmetic preparation advantageously contains less than 10 aromatic substances or perfumes and particularly preferably no aromatic substances or perfumes.
d) The use of octyl salicylate for perfuming and/or odor stabilization of cosmetic preparations containing 1,3-dihydroxyacetone and characterized according to the packaging information as “unscented,” “fragrance-free,” “odorless” or with a synonymous description.
e) A cosmetic of
   i. A cosmetic preparation containing octyl salicylate and 1,3-dihydroxyacetone,
   ii. A packaging container and
   iii. A packaging label characterizing the cosmetic preparation as “unscented,” “fragrance-free,” “odorless” or a similar designation.

One skilled in the art is familiar with preparations that disclose a combination of octyl salicylate and 1,3-dihydroxyacetone. For example, WO 02/28359 and WO 97/25970 describe combinations of 1,3-dihydroxyacetone, antioxidants and UV sunscreen filters. However, these documents could not show the way to the present invention. The teaching according to the invention according to which octyl salicylate suppresses the perception of the odor of decomposition products of the 1,3-dihydroxyacetone for a prolonged period is not disclosed in any of the documents of the prior art.

The octyl salicylate (actually salicylic acid-2-ethylhexylester, CAS no. 118-60-5) is a UVB sunscreen filter approved for cosmetic preparations, which can be obtained, inter alia, under the trade name Escalol 587 (ISP) or Neo Heliopan OS (Symrise). It is characterized by the following structure:

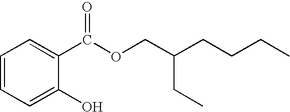

Octyl salicylate is advantageously used according to the invention in a concentration of from 0.01 to 5.0% by weight, based on the total weight of the cosmetic preparation.

The 1,3-dihydroxyacetone according to the invention is advantageously used according to the invention in a concentration of from 0.1 to 25.0% by weight, based on the total weight of the cosmetic preparation.

In particular, it is preferred according to the invention if in the cosmetic according to the invention or with the use according to the invention, the weight ratio of octyl salicylate to 1,3-dihydroxyacetone in the preparations is from 1:10 to 1:5.

Preparations according to the invention may be present in various forms. Thus, for example, they may be a solution, an anhydrous preparation, an emulsion or microemulsion of the water-in-oil (W/O) type or of the oil-in-water (O/W) type, a multiple emulsion, for example of the water-in-oil-in-water (W/O/W) type, a gel, a solid stick, an ointment or else an aerosol.

Accordingly, the use according to the invention in cosmetic preparations where the latter are in the form of a multiple emulsion, microemulsion, Pickering emulsion, mousse, foam or sprayable emulsion and the like is in accordance with the invention.

Emulsions according to the invention are advantageous and comprise, for example, the specified fats, oils, waxes and other fatty bodies, and water and an emulsifier, as is customarily used for such a type of formulation.

The lipid phase can advantageously be chosen from the following group of substances:
mineral oils, mineral waxes;
oils, such as triglycerides of capric acid or of caprylic acid, and also natural oils, such as, for example, castor oil,
fats, waxes and other natural and synthetic fatty bodies, preferably esters of fatty acids with alcohols of low carbon number, e.g. with isopropanol, propylene glycol or glycerol, or esters of fatty alcohols with alkanoic acids of low carbon number or with fatty acids;
alkyl benzoates; and
silicone oils, such as dimethylpolysiloxanes, diethylpolysiloxanes, diphenylpolysiloxanes, and mixed forms thereof.

The oil phase of the emulsions, oleogels or hydrodispersions or lipodispersions for the purposes of the present invention is advantageously chosen from the group of esters of saturated and/or unsaturated, branched and/or unbranched alkanecarboxylic acids of a chain length of from 3 to 30 C atoms and saturated and/or unsaturated, branched and/or unbranched alcohols of a chain length of from 3 to 30 C atoms, from the group of esters of aromatic carboxylic acids and saturated and/or unsaturated, branched and/or unbranched alcohols of a chain length of from 3 to 30 C atoms. Such ester oils can then advantageously be chosen from the group isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate, and synthetic, semisynthetic and natural mixtures of such esters, e.g., jojoba oil.

In addition, the oil phase can advantageously be chosen from the group of branched and unbranched hydrocarbons and hydrocarbon waxes, silicone oils, dialkyl ethers, the group of saturated or unsaturated, branched or unbranched alcohols, and fatty acid triglycerides, namely the triglycerol esters of saturated and/or unsaturated, branched and/or unbranched alkanecarboxylic acids of a chain length of from 8 to 24, in particular 12-18, C atoms. The fatty acid triglycerides can, for example, be chosen advantageously from the group of synthetic, semisynthetic and natural oils, e.g. olive oil, sunflower oil, soya oil, peanut oil, rapeseed oil, almond oil, palm oil, coconut oil, palm kernel oil and the like.

Any mixtures of such oil and wax components can also be used advantageously for the purposes of the present invention. In some cases, it may also be advantageous to use waxes, for example cetyl palmitate, as the sole lipid component of the oil phase.

The oil phase is advantageously chosen from the group consisting of 2-ethylhexyl isostearate, octyidodecanol, isotridecyl isononanoate, isoeicosane, 2-ethylhexyl cocoate, C₁₂₋₁₅-alkyl benzoate, caprylic/capric triglyceride, dicaprylyl ether.

Mixtures of C₁₂₋₁₅-alkyl benzoate and 2-ethylhexyl isostearate, mixtures of C₁₂₋₁₅-alkyl benzoate and isotridecyl isononanoate and mixtures of C₁₂₋₁₅-alkyl benzoate, 2-ethylhexyl isostearate and isotridecyl isononanoate are particularly advantageous.

Of the hydrocarbons, paraffin oil, squalane and squalene are to be used advantageously for the purposes of the present invention.

The oil phase can also advantageously have a content of cyclic or linear silicone oils, or consist entirely of such oils, although it is preferred, apart from the silicone oil or the silicone oils, to use an additional content of other oil phase components.

Cyclomethicone (octamethylcyclotetrasiloxane) is advantageously used as silicone oil to be used according to the invention. Other silicone oils, however, are also to be used advantageously for the purposes of the present invention, for example hexamethylcyclotrisiloxane, polydimethylsiloxane, poly-(methylphenylsiloxane).

Also particularly advantageous are mixtures of cyclomethicone and isotridecyl isononanoate, and of cyclomethicone and 2-ethylhexyl isostearate.

The aqueous phase of the preparations according to the invention optionally advantageously comprises alcohols, diols or polyols of low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, and also alcohols of low carbon number, e.g. ethanol, isopropanol, 1,2-propanediol, glycerol and in particular one or more thickeners which can be chosen advantageously from the group of silicon dioxide, aluminum silicates, polysaccharides and derivatives thereof, e.g. hyaluronic acid, xanthan gum, hydroxypropylmethylcellulose, particularly advantageously from the group of polyacrylates, preferably a polyacrylate from the group of so-called Carbopols, for example Carbopol grades 980, 981, 1382, 2984, 5984, in each case individually or in combination.

In particular, mixtures of the abovementioned solvents are used. In the case of alcoholic solvents, water may be a further constituent.

Emulsions according to the invention are advantageous and contain, for example, the specified fats, oils, waxes and other fatty bodies, and water and an emulsifier, as is customarily used for such a type of formulation.

Gels according to the invention usually contain alcohols of low C number, e.g., ethanol, isopropanol, 1,2-propanediol, glycerol and water or an abovementioned oil in the presence of a thickener which, in the case of oily-alcoholic gels, is preferably silicon dioxide or an aluminum silicate, and in the case of aqueous-alcoholic or alcoholic gels is preferably a polyacrylate.

The cosmetic and dermatological preparations according to the invention can contain cosmetic auxiliaries as are customarily used in such preparations, e.g., preservatives, bactericides, perfumes, substances for preventing foaming, dyes, pigments which have a coloring effect, thickeners, surface-active substances, emulsifiers, softening, moisturizing and/or humectant substances, fats, oils, waxes or other customary constituents of a cosmetic or dermatological formulation, such as alcohols, polyols, polymers, foam stabilizers, electrolytes, organic solvents or silicone derivatives.

In particular, active ingredient combinations used according to the invention can also be combined with other antioxidants and/or free-radical scavengers.

Such antioxidants are advantageously chosen from the group consisting of amino acids (e.g., glycine, histidine, tyrosine, tryptophan) and derivatives thereof, imidazoles (e.g., urocanic acid) and derivatives thereof, peptides, such as D,L-carnosine, D-carnosine, L-carnosine and derivatives thereof (e.g., anserine), carotenoids, carotenes (e.g., α-carotene, β-carotene, lycopene) and derivatives thereof, chlorogenic acid and derivatives thereof, lipoic acid and derivatives thereof (e.g., dihydrolipoic acid), aurothioglucose, propylthiouracil and other thiols (e.g., thioredoxin, glutathione, cysteine, cystine, cystamine and the glycosyl, N-acetyl, methyl, ethyl, propyl, amyl, butyl and lauryl, palmitoyl, oleyl, y-linoleyl, cholesteryl and glyceryl esters thereof), and salts thereof, dilauryl thiodipropionate, distearyl thiodipropionate, thiodipropionic acid and derivatives (esters, ethers, peptides, lipids, nucleotides, nucleosides and salts) thereof, and sulphoximine compounds (e.g., buthionine sulphoximines, homocysteine sulphoximine, buthionine sulphones, penta-, hexa-, heptathionine sulphoximine) in very low tolerated doses (e.g., pmol to μmol/kg), and also (metal) chelating agents (e.g. α-hydroxy fatty acids, palmitic acid, phytic acid, phytin, lactoferrin), α-hydroxy acids (e.g., citric acid, lactic acid, malic acid), humic acid, bile acid, bile extracts, bilirubin, biliverdin, EDTA, EGTA and derivatives thereof, unsaturated fatty acids and derivatives thereof (e.g., γ-linolenic acid, linoleic acid, oleic acid), folic acid and derivatives thereof, ubiquinone and ubiquinol and derivatives thereof, vitamin C and derivatives (e.g., ascorbyl palmitate, Mg-ascorbyl phosphate, ascorbyl acetate), tocopherols and derivatives (e.g., vitamin E acetate), vitamin A and derivatives (vitamin A palmitate), and coniferyl benzoate of benzoin resin, rutinic acid and derivatives thereof, butylated hydroxytoluene, butylated hydroxyanisol, nordihydroguaiacic acid, nordihydroguaiaretic acid, trihydroxybutyrophenone, uric acid and derivatives thereof, mannose and derivatives thereof, sesamol, sesamolin, zinc and derivatives thereof (e.g., ZnO, ZnSO₄), selenium and derivatives thereof (e.g., selenomethionine), stilbenes and derivatives thereof (e.g., stilbene oxide, trans-stilbene oxide) and the derivatives of these active ingredients mentioned which are suitable according to the invention (salts, esters, ethers, sugars, nucleotides, nucleosides, peptides and lipids).

The amount of the abovementioned antioxidants (one or more compounds) in the preparations is preferably from 0.001 to 30% by weight, particularly preferably 0.05-20% by weight, in particular 1-10% by weight, based on the total weight of the preparation.

If vitamin E and/or derivatives thereof are the additional antioxidant or antioxidants, it is advantageous to choose their respective concentrations from the range from 0.001-10% by weight, based on the total weight of the formulation.

If vitamin A or vitamin A derivatives, or carotenes or derivatives thereof are the additional antioxidant or antioxidants, it is advantageous to choose their respective concentrations from the range from 0.001-10% by weight, based on the total weight of the formulation.

Water-soluble antioxidants, such as vitamins, e.g., ascorbic acid and derivatives thereof can be used particularly advantageously for the purposes of the present invention.

Preparations according to the invention can advantageously also comprise substances which absorb UV radiation in the UVB region, where the total amount of the filter substances is, for example, from 0.1% by weight to 30% by weight, preferably from 0.5 to 10% by weight, in particular from 1.0 to 6.0% by weight, based on the total weight of the preparations, in order to provide cosmetic preparations which protect the hair and/or the skin from the entire range of ultraviolet radiation. They can also serve as sunscreen compositions for the hair or the skin.

If the preparations according to the invention comprise further UVB filter substances, these may be oil-soluble or water-soluble. Oil-soluble UVB filters advantageous according to the invention are, for example:
3-benzylidenecamphor derivatives, preferably 3-(4-methylbenzylidene)camphor, 3-benzylidenecamphor;
4-aminobenzoic acid derivatives, preferably 2-ethylhexyl 4-(dimethylamino)benzoate, amyl 4-(dimethylamino)benzoate;
esters of cinnamic acid, preferably 2-ethylhexyl 4-methoxycinnamate, isopentyl 4-methoxycinnamate;
esters of salicylic acid, preferably 4-isopropylbenzyl salicylate, homomenthyl salicylate;
derivatives of benzophenone, preferably 2-hydroxy-4-methoxybenzophenone, 2-hydroxy-4-methoxy-4′-methylbenzophenone, 2,2′-dihydroxy-4-methoxybenzophenone;
esters of benzalmalonic acid, preferably di(2-ethylhexyl) 4-methoxybenzalmalonate, 2,4,6-trianilino(p-carbo-2′-ethyl-1′-hexyloxy)-1,3,5-triazine.

Advantageous water-soluble UVB filters are, for example:
salts of 2-phenylbenzimidazole-5-sulphonic acid, such as its sodium, potassium or its triethanolammonium salt, and the sulphonic acid itself;
sulfonic acid derivatives of benzophenones, preferably 2-hydroxy-4-methoxybenzophenone-5-sulphonic acid and its salts; and
sulfonic acid derivatives of 3-benzylidenecamphor, such as, for example, 4-(2-oxo-3-bornylidenemethyl)benzenesulphonic acid, 2-methyl-5-(2-oxo-3-bornylidenemethyl)sulphonic acid and its salts, and 1,4-di(2-oxo-10-sulpho-3-bornylidenemethyl)benzene and salts thereof (the corresponding 10-sulphato compounds, for example the corresponding sodium, potassium or triethanolammonium salt), also referred to as benzene-1,4-di(2-oxo-3-bornylidenemethyl-10-sulphonic acid.

The list of specified UVB filters which can be used in combination with the active ingredient combinations according to the invention is of course not intended to be limiting.

It may also be advantageous to use in the preparations according to the invention UVA filters which are customarily present in cosmetic preparations. These substances are preferably derivatives of dibenzoylmethane, in particular 1-(4′-tert-butylphenyl)-3-(4′-methoxyphenyl)propane-1,3-dione and 1-phenyl-3-(4′-isopropylphenyl)propane-1,3-dione. These combinations or preparations that contain these combinations are also the subject matter of the invention. It is possible to use the amounts used for the UVB combination.

Cosmetic preparations according to the invention can also contain inorganic pigments that are usually used in cosmetics to protect the skin from UV radiation. These are oxides of titanium, zinc, zirconium, silicon, manganese, cerium, and mixtures of such oxides, and modifications in which the oxides are the active agents. These are particularly preferably pigments based on titanium dioxide.

These combinations of UVA filter and pigment or preparations containing these combinations are subject matter of the invention as well. The amounts specified for the abovementioned combinations may be used.

Moisturizers is the term used to refer to substances or mixtures of substances which impart to cosmetic or dermatological preparations the property, following application or distribution on the surface of the skin, of reducing moisture release by the horny layer (also called transepidermal water loss (TEWL)) and/or of positively influencing hydration of the horny layer.

Within the meaning of the present invention, advantageous moisturizers are, e.g., glycerol, lactic acid, pyrrolidonecarboxylic acid and urea. In addition, it is particularly advantageous to use polymeric moisturizers from the group of water-soluble and/or in water-swellable or and/or water-gellable polysaccharides. Hyaluronic acid, chitosan and/or a fucose-rich polysaccharide, which is filed in the Chemical Abstracts under the Registry number 178463-23-5 and which is available, for example, under the name Fucogel®1000 from SOLABIA S.A., for example, are particularly advantageous.

Preferred embodiments of the present invention are characterized in that they contain one or more moisturizers chosen from the group glycerol, lactic acid, pyrrolidonecarboxylic acid and urea in a total concentration of up to 0.1-15% by weight, based on the total weight of the emulsion.

Advantageous embodiments of the present invention are further characterized in that the cosmetic preparation contains cyclodextrins and/or derivatives thereof.

“Cyclodextrins and/or derivatives thereof” means both cyclodextrins with a different number of glucose units in the ring molecule and derivatives of these compounds.

It is advantageous in accordance with the invention to use native, polar and/or apolar substituted α-, β-, and γ-cyclodextrins. These include preferably, but not exclusively, methyl-, especially random-methyl-β-cyclodextrin, ethyl- and also hydroxypropyl-cyclodextrins. The cyclodextrin species particularly preferred according to the invention are γ-cylcodextrin and hydroxypropyl-β-cyclodextrin.

It is preferred according to the invention if the cosmetic according to the invention or a cosmetic in which the use according to the invention is realized, is characterized in that the cosmetic preparation is a sun block.

It is preferred according to the invention if the cosmetic according to the invention or a cosmetic in which the use according to the invention is realized, is characterized in that the cosmetic preparation is a self-tanning preparation for tanning the skin and or the integumentary appendages (e.g., hair, nails) and is particularly well tolerated by the skin (“hypoallergenic”).

The following examples are designed to clarify the present invention without restricting it. All amounts, proportions and percentages refer to the weight and the total amount or to the total weight of the preparations, unless stated otherwise.

### W/O Emulsions

| | 1 | 2 | 3 | 4 | 5 |
|---|---|---|---|---|---|
| Triglycerin diisostearate | 1.0 | 0.5 | 0.25 | 2.0 | 3.0 |
| Diglycerin | 1.0 | 1.5 | 1.75 | 3.0 | 2.0 |
| dipolyhydroxystearate | | | | | |
| Paraffin oil | 12.5 | 10.0 | 8.0 | 5.0 | 7.5 |
| Petrolatum | 8.0 | 6.0 | 5.0 | 2.0 | 2.5 |
| Hydrogenated | 2.0 | 1.0 | 2.5 | 5.0 | 0.25 |
| cocoglyceride | | | | | |
| Decyl oleate | 0.5 | 0.75 | 1.0 | 2.0 | 0.25 |
| Octyldodecanol | 0.5 | 1.0 | 0.75 | 3.0 | 0.25 |
| Aluminum stearate | 0.4 | 0.3 | 0.6 | 1.0 | 0.05 |
| Dicaprylyl carbonate | 0.1 | 0.05 | 0.15 | 0.5 | 1.0 |
| Hydrogenated castor oil | 0.5 | 0.75 | 1.0 | 2.5 | 5.0 |
| Octyl salicylate | 0.5 | 1.0 | 1.5 | 2.0 | 3.5 |
| Dihydroxyacetone | 2.5 | 5.0 | 7.5 | 20.0 | 17.5 |
| Magnesium sulfate | 0.5 | 0.6 | 0.5 | 0.7 | 1.0 |
| Glycerol | 3.0 | 5.0 | 10.0 | 15.0 | 1.5 |
| Citric acid | 0.2 | 0.1 | 0.2 | 0.3 | 1.0 |
| Sodium citrate | 0.2 | 0.05 | 0.4 | 0.3 | 2.0 |
| Ethanol | 2.0 | — | 5.0 | — | — |
| Capric/caprylic | 2.0 | 2.5 | 3.0 | 5.0 | 0.5 |
| acid triglyceride | | | | | |
| Potassium sorbate | 0.04 | 0.15 | 0.05 | 0.03 | 0.4 |
| Benzyl alcohol | 0.3 | 0.4 | 0.25 | 0.15 | — |
| Water | ad 100 | ad 100 | ad 100 | ad 100 | ad 100 |

### W/O Emulsions

| | 6 | 7 | 8 | 9 | 10 |
|---|---|---|---|---|---|
| PEG 30 | — | 0.5 | 0.25 | — | 3.0 |
| Dipolyhydroxystearate | | | | | |
| Lanolin alcohol | 1.0 | 1.5 | 1.75 | 3.0 | — |
| Paraffin oil | 12.5 | 10.0 | 8.0 | 5.0 | 7.5 |
| Petrolatum | 8.0 | 6.0 | 5.0 | 4.0 | 2.5 |
| Hydrogenated | 2.0 | 1.0 | 2.5 | 5.0 | 0.25 |
| cocoglyceride | | | | | |
| Hydrogenated | 0.5 | 0.75 | 1.0 | 2.0 | 0.25 |
| polyisobutene | | | | | |
| Octyldodecanol | 0.5 | 1.0 | 0.75 | 3.0 | 0.25 |
| Aluminum stearate | 0.4 | 0.3 | 0.6 | 1.0 | 0.05 |
| Dicaprylyl carbonate | 0.1 | 0.05 | 0.15 | 0.5 | 1.0 |
| Hydrogenated castor oil | 0.5 | 0.75 | 1.0 | 2.5 | 5.0 |
| Octyl salicylate | 0.5 | 1.0 | 1.5 | 2.0 | 3.5 |
| Dihydroxyacetone | 2.5 | 5.0 | 7.5 | 20.0 | 17.5 |
| Magnesium sulfate | 0.5 | 0.6 | 0.5 | 0.7 | 1.0 |
| Glycerol | 3.0 | 5.0 | 10.0 | 15.0 | 1.5 |
| Citric acid | 0.2 | 0.1 | 0.2 | 0.3 | 1.0 |
| 1,3-Butylene glycol | 2.0 | — | 5.0 | — | — |
| Capric/caprylic | 2.0 | 2.5 | 3.0 | 5.0 | 0.5 |
| acid triglyceride | | | | | |
| Sodium dehydracet | — | — | 0.05 | — | — |
| Potassium sorbate | 0.3 | 0.4 | 0.25 | 0.15 | — |
| Talc | — | — | 0.05 | — | 0.1 |
| Water | ad 100 | ad 100 | ad 100 | ad 100 | ad 100 |

### W/S Emulsion

| | 11 | 12 | 13 | 14 | 15 |
|---|---|---|---|---|---|
| Cetyl PEG/PPG-10/1 | 1.0 | — | — | 3.0 | 5.0 |
| dimethicone | | | | | |
| Cyclomethicone + PEG/ | 10.0 | 12.5 | 25 | — | — |
| PPG-18/18 dimethicone | | | | | |
| (90:10) | | | | | |
| Cyclomethicone | 12.5 | 15 | 28.0 | 25.0 | 17.5 |
| Dimethicone | 5.0 | 13.0 | 5.0 | 12.0 | 15.0 |
| Hydrogenated | 0.5 | 0.75 | 1.0 | 2.0 | 0.25 |
| polyisobutene | | | | | |
| Octyldodecanol | 0.5 | 1.0 | 0.75 | 3.0 | 0.25 |
| Panthenol | 0.5 | 1.0 | 0.75 | 0.25 | 0.1 |
| Sodium chloride | 2.0 | 0.6 | 2.5 | 0.7 | 1.0 |
| Glycerol | 3.0 | 5.0 | 10.0 | 15.0 | 1.5 |
| Octyl salicylate | 0.5 | 1.0 | 1.5 | 2.0 | 3.5 |
| Dihydroxyacetone | 2.5 | 5.0 | 7.5 | 20.0 | 17.5 |
| Citric acid | 0.2 | 0.1 | 0.2 | 0.3 | 1.0 |
| Sodium citrate | 1.0 | 0.1 | 0.4 | 0.9 | 2.5 |
| Potassium sorbate | 0.4 | 0.1 | 0.05 | 0.3 | 0.4 |
| Cetyldimethicone | 0.5 | — | 0.7 | — | — |
| Benzyl alcohol | — | — | 0.05 | — | 0.1 |
| Water | ad | ad | ad | ad | ad |
| | 100 | 100 | 100 | 100 | 100 |

### W/S Emulsions

| | 16 | 17 | 18 | 19 | 20 |
|---|---|---|---|---|---|
| Cetyl PEG/PPG-10/1 | 1.0 | — | — | 3.0 | 5.0 |
| dimethicone | | | | | |
| Cyclomethicone + PEG/ | 10.0 | 12.5 | 25 | — | — |
| PPG-18/18 dimethicone | | | | | |
| (90:10) | | | | | |
| Cyclomethicone | 12.5 | 15 | 18.0 | 15.0 | 11.5 |
| Dimethicone | 5.0 | 13.0 | 5.0 | 12.0 | 5.0 |
| Hydrogenated | 0.5 | 0.75 | 1.0 | 2.0 | 0.25 |
| polyisobutene | | | | | |
| Octyldodecanol | 0.5 | 1.0 | 0.75 | 3.0 | 0.25 |
| Panthenol | 0.5 | 1.0 | 0.75 | 0.25 | 0.1 |
| Sodium chloride | 2.0 | 0.6 | 2.5 | 0.7 | 1.0 |
| Glycerol | 3.0 | 5.0 | 10.0 | 15.0 | 1.5 |
| Lactic acid | 0.2 | 0.1 | 0.2 | — | 13 |
| Sodium lactate | 0.2 | 1.0 | 0.05 | — | — |
| Octyl salicylate | 0.5 | 1.0 | 1.5 | 2.0 | 3.5 |
| Dihydroxyacetone | 2.5 | 5.0 | 7.5 | 20.0 | 17.5 |
| Stearyl dimethicone | 0.5 | — | 0.7 | — | — |
| Dehydroacetic acid | — | — | 0.05 | — | 0.1 |
| Modified starch | — | 2.5 | — | 0.15 | — |
| Water | ad | ad | ad | ad | ad |
| | 100 | 100 | 100 | 100 | 100 |

### WIO Emulsions

| | 21 | 22 | 23 | 24 | 25 |
|---|---|---|---|---|---|
| PEG-22 dodecyl glycol | 5.0 | 1.5 | 0.25 | — | 3.0 |
| copolymer | | | | | |
| PEG-45 dodecyl glycol | 1.0 | 1.5 | 1.75 | 3.0 | — |
| polymer | | | | | |
| Paraffin oil | 12.5 | 10.0 | 8.0 | 5.0 | 17.5 |
| Isopropyl stearate | 8.0 | 6.0 | 5.0 | 12.0 | 2.5 |
| Hydrogenated | 2.0 | 1.0 | 2.5 | 5.0 | 0.25 |
| cocoglycerides | | | | | |
| Oil of evening primrose | 0.5 | 0.75 | 1.0 | 2.0 | 0.25 |
| Octyldodecanol | 0.5 | 1.0 | 0.75 | 3.0 | 0.25 |
| Aluminum stearate | 0.4 | 0.3 | 0.6 | 1.0 | 0.05 |
| Dicaprylyl carbonate | 0.1 | 0.05 | 0.15 | 0.5 | 1.0 |
| Hydrogenated castor oil | 0.5 | 0.75 | 1.0 | 2.5 | 5.0 |
| Octyl salicylate | 0.5 | 1.0 | 1.5 | 2.0 | 3.5 |
| Dihydroxyacetone | 2.5 | 5.0 | 7.5 | 20.0 | 17.5 |
| Magnesium sulfate | 0.5 | 0.6 | 0.5 | 0.7 | 1.0 |
| Glycerol | 3.0 | 5.0 | 10.0 | 15.0 | 1.5 |
| Sodium citrate | 0.2 | 0.1 | — | — | — |
| Citric acid | 0.2 | 0.1 | — | — | — |
| 1,3-Butylene glycol | 2.0 | — | 5.0 | — | — |
| Capric/caprylic acid | 2.0 | 2.5 | 3.0 | 5.0 | 0.5 |
| triglyceride | | | | | |
| Potassium sorbate | 0.4 | 0.15 | 0.05 | 0.3 | 0.4 |
| Benzyl alcohol | — | — | 0.05 | — | 0.1 |
| Water | ad 100 | ad 100 | ad 100 | ad 100 | ad 100 |

### W/O Emulsions

| | 26 | 27 | 28 | 29 | 30 |
|---|---|---|---|---|---|
| Polyglyceryl-2- | 3.0 | — | 0.25 | — | 3.0 |
| dipolyhydroxystearate | | | | | |
| Polyglyceryl-3 diisostearate | 1.0 | 3.5 | 1.75 | 2.5 | — |
| PEG-40 sorbitan isostearate | — | 2.5 | 0.5 | 3.5 | 3.0 |
| Paraffin oil | 12.5 | 10.0 | 8.0 | 5.0 | 17.5 |
| Isopropylstearate | 8.0 | 6.0 | 5.0 | 12.0 | 2.5 |
| Hydrogenated | 2.0 | 1.0 | 2.5 | 5.0 | 0.25 |
| cocoglycerides | | | | | |
| Isopropyl palmitate | 0.5 | 1.0 | 0.75 | 3.0 | 0.25 |
| Dicaprylyl carbonate | 0.1 | 0.05 | 0.15 | 0.5 | 1.0 |
| Hydrogenated castor oil | 0.5 | 0.75 | 1.0 | 2.5 | 5.0 |
| Octyl salicylate | 0.5 | 1.0 | 1.5 | 2.0 | 3.5 |
| Dihydroxyacetone | 2.5 | 5.0 | 7.5 | 20.0 | 17.5 |
| Magnesium sulfate | 0.5 | 0.6 | 0.5 | 0.7 | 1.0 |
| Glycerol | 3.0 | 5.0 | 10.0 | 15.0 | 1.5 |
| Citric acid | 0.2 | 0.1 | 0.1 | 0.3 | 1.0 |
| Sodium citrate | 0.2 | 0.3 | 0.2 | 1.5 | 0.8 |
| Capric/caprylic acid | 2.0 | 2.5 | 3.0 | 5.0 | 0.5 |
| triglyceride | | | | | |
| Potassium sorbate | 0.24 | 0.15 | 0.05 | 0.3 | 0.4 |
| Water | ad | ad | ad | ad | ad |
| | 100 | 100 | 100 | 100 | 100 |

### Silicone in Water Emulsion

| | 31 | 32 | 33 | 34 | 35 |
|---|---|---|---|---|---|
| Dimethicone copolyol | 1.0 | 2.0 | 8.0 | 3.0 | 5.0 |
| Capric/caprylic | | | | | |
| triglyceride | | | | | |
| Cyclomethicone | 12.5 | 15 | 25.0 | 10.0 | 7.5 |
| Dimethicone | 5.0 | 15.0 | 5.0 | 12.0 | 15.0 |
| Mineral oil | 0.5 | 0.75 | 1.0 | 2.0 | 0.25 |
| Phenyltrimethicone | 0.5 | 1.0 | 0.75 | 3.0 | 0.25 |
| Glycerol | 5.0 | 7.5 | 10.0 | 3.0 | 1.0 |
| Xanthan Gum | — | 0.1 | — | 0.25 | 1.0 |
| Panthenol | 0.5 | 1.0 | 0.75 | 0.25 | 0.1 |
| Methyl paraben | 0.4 | 0.1 | 0.05 | 0.3 | 0.4 |
| Octyl salicylate | 0.5 | 1.0 | 1.5 | 2.0 | 3.5 |
| Dihydroxyacetone | 2.5 | 5.0 | 7.5 | 20.0 | 17.5 |
| Propyl paraben | 0.3 | 0.4 | 0.25 | 0.15 | — |
| Iodopropynyl | — | — | 0.05 | — | 0.1 |
| butylcarbamate | | | | | |
| Water | ad 100 | ad 100 | ad 100 | ad 100 | ad 100 |

### O/W Emulsion with Tapioca

| | 36 | 37 | 38 | 39 | 40 |
|---|---|---|---|---|---|
| Glyceryl stearate | 1.0 | — | — | 0.5 | 0.25 |
| Polyethylene | 10.0 | — | 5 | — | — |
| glycol(40)stearate | | | | | |
| Triglycerin | — | 5.5 | — | — | 2.5 |
| methylglucose distearate | | | | | |
| Sorbitan stearate | — | 1.5 | 3 | — | — |
| Cyclomethicone | 2.5 | 15 | 8.0 | 5.0 | 7.5 |
| Dimethicone | 5.0 | 3.0 | 5.0 | 2.0 | 5.0 |
| Behenyl alcohol | 1 | — | 2 | 1 | — |
| Stearyl alcohol | — | 1 | — | 1 | — |
| Cetylstearyl alcohol | — | — | 1 | 1 | — |
| Octyl salicylate | 0.5 | 1.0 | 1.5 | 2.0 | 3.5 |
| Dihydroxyacetone | 2.5 | 5.0 | 7.5 | 20.0 | 17.5 |
| Hydrogenated | 0.5 | 0.75 | 1.0 | 2.0 | 0.25 |
| polyisobutene | | | | | |
| Octyldodecanol | 0.5 | 1.0 | 0.75 | 3.0 | 0.25 |
| Methyl paraben | 0.4 | 0.1 | 0.05 | 0.3 | 0.4 |
| Propyl paraben | 0.3 | 0.4 | 0.25 | 0.15 | — |
| Iodopropynyl | — | — | 0.05 | — | 0.1 |
| butylcarbamate | | | | | |
| Glycerol | 5 | 10 | 3 | 15 | 7.5 |
| Modified starch | — | 2.5 | — | 0.15 | 13 |
| Water | ad 100 | ad 100 | ad 100 | ad 100 | ad 100 |

### O/W Emulsion

| | 41 | 42 | 43 | 44 | 45 |
|---|---|---|---|---|---|
| Polyethyl- | 1 | — | 2.5 | 2 | 1.5 |
| eneglycol(21)stearylether | | | | | |
| Polyethyl- | 1 | — | 5.5 | 3 | 7.5 |
| eneglycol(2)stearylether | | | | | |
| Cetearyl glucoside | — | 8 | — | — | — |
| Behenyl alcohol | 3 | 2 | — | 1 | — |
| Stearyl alcohol | 3 | 2 | — | 2 | — |
| Cetylstearyl alcohol | 3 | 4 | — | — | 2 |
| Hydrogenated | 0.5 | 0.75 | 1.0 | 2.0 | 0.25 |
| polyisobutene | | | | | |
| Octyl salicylate | 0.5 | 1.0 | 1.5 | 2.0 | 3.5 |
| Dihydroxyacetone | 2.5 | 5.0 | 7.5 | 20.0 | 17.5 |
| Octyldodecanol | 0.5 | 1.0 | 0.75 | 3.0 | 0.25 |
| Glycerol | 5 | 10 | 15 | 3 | 7.5 |
| Panthenol | 0.5 | 1.0 | 0.75 | 0.25 | 0.1 |
| Methyl paraben | 0.4 | 0.1 | 0.05 | 0.3 | 0.4 |
| Propyl paraben | 0.3 | 0.4 | 0.25 | 0.15 | — |
| Iodopropynyl | — | — | 0.05 | — | 0.1 |
| butylcarbamate | | | | | |
| Modified starch | 0.5 | — | — | 0.15 | — |
| Water | ad 100 | ad 100 | ad 100 | ad 100 | ad 100 |

### O/W Emulsion

| | 46 | 47 | 48 | 49 | 50 |
|---|---|---|---|---|---|
| Glycerylsterate citrate | 1.0 | 0.5 | 0.1 | 0.5 | 0.3 |
| Polyethyl- | 10.0 | 1.0 | 5 | — | — |
| eneglycol(20)cetearyl- | | | | | |
| ether | | | | | |
| Triglycerinmethylglucose | — | — | — | — | 2.5 |
| distearate | | | | | |
| Ethylbutylacetyl | 5 | — | 15 | 20 | — |
| aminopropionate | | | | | |
| Dimethicone | 0.5 | 3.0 | 0.75 | 1.5 | 0.2 |
| Behenyl alcohol | 1 | — | 2 | 1 | 0.2 |
| Dicaprylylcarbonate | 3 | 5 | 10 | 15 | 5 |
| Stearyl alcohol | — | — | — | 1 | 0.2 |
| Cetylstearyl alcohol | — | — | 1 | 1 | 0.2 |
| Tocopherol | 0.5 | 0.5 | 0.75 | 0.25 | 0.1 |
| Octyldodecanol | 0.5 | — | 0.75 | 3.0 | 0.25 |
| Panthenol | 0.5 | — | 0.75 | 0.25 | 0.1 |
| Octyl salicylate | 0.5 | 1.0 | 1.5 | 2.0 | 3.5 |
| Dihydroxyacetone | 2.5 | 5.0 | 7.5 | 20.0 | 17.5 |
| Carbomer | 0.05 | 0.35 | 0.15 | 0.1 | — |
| Capric/caprylic | 1 | 5 | 3 | 5 | 10 |
| triglyceride | | | | | |
| Methyl paraben | 0.4 | 0.3 | 0.05 | 0.3 | 0.4 |
| Propyl paraben | 0.3 | — | 0.25 | 0.15 | — |
| Iodopropynyl | — | — | 0.05 | — | 0.1 |
| butylcarbamate | | | | | |
| Phenoxyethanol | — | 0.5 | — | 0.15 | — |
| Sorbitol | 10 | — | — | 5 | — |
| Butylene glycol | — | — | — | 5 | 10 |
| Propylene glycol | — | — | 10 | 5 | — |
| Glycerol | — | 7.5 | — | — | — |
| Water | ad 100 | ad 100 | ad 100 | ad 100 | ad 100 |

The present application is based on German Patent Application No. 10 002 170.8, filed Jan. 12, 2004, the entire disclosure whereof is sly incorporated by reference herein.

1.-10. (canceled)

11. A method of perfuming and/or improving odor of a cosmetic preparation which comprises 1,3-dihydroxyacetone and decomposition products of 1,3-dihydroxyacetone, wherein the method comprises incorporating in the preparation an effective amount of octyl salicylate.

12. The method of claim 11, wherein the preparation does not contain fragrances and perfumes.

13. The method of claim 11, wherein the preparation is hypoallergenic.

14. The method of claim 11, wherein the preparation is associated with a packaging information which characterizes the preparation as at least one of “fragrance-free,” “unscented”, “odorless” or a synonymous description.

15. The method of claim 11, wherein a weight ratio of octyl salicylate to 1,3-dihydroxyacetone in the preparation is from 1:10 to 1:5.

16. The method of claim 11, wherein the preparation further comprises at least one of a cyclodextrin and a derivative thereof.

17. The method of claim 11, wherein the preparation comprises a sunscreen.

18. The method of claim 11, wherein the preparation comprises a self-tanning preparation.

19. A method of masking odor and/or stabilizing odor in a cosmetic preparation which comprises 1,3-dihydroxyacetone and decomposition products thereof, wherein the method comprises incorporating in the preparation octyl salicylate in an amount which is effective to at least one of mask and stabilize odor which is due to the decomposition products.

20. A cosmetic preparation comprising 1,3-dihydroxyacetone, wherein the preparation further comprises decomposition products of 1,3-dihydroxyacetone and octyl salicylate in an amount which is effective to at least one of mask and stabilize an odor which is due to the decomposition products of 1,3-dihydroxyacetone.

21. The preparation of claim 20, wherein the preparation is substantially free of fragrances and perfumes.

22. The preparation of claim 20, wherein the preparation is hypoallergenic.

23. The preparation of claim 20, wherein the preparation is associated with a packaging information which characterizes the preparation as at least one of “fragrance-free,” “unscented”, “odorless” or a synonymous description.

24. The preparation of claim 20, wherein a weight ratio of octyl salicylate to 1,3-dihydroxyacetone is from 1:10 to 1:5.

25. The preparation of claim 20, wherein the preparation further comprises at least one of a cyclodextrin and a derivative thereof.

26. The preparation of claim 20, wherein the preparation comprises a sunscreen.

27. The preparation of claim 20, wherein the preparation comprises a self-tanning preparation.

28. The preparation of claim 21, wherein the preparation is hypoallergenic.

29. The preparation of claim 21, wherein the preparation further comprises at least one of a cyclodextrin and a derivative thereof.

30. The preparation of claim 21, wherein a weight ratio of octyl salicylateto 1,3-dihydroxyacetone is from 1:10 to 1:5.
